(19) United States
(12) Patent Application Publication
(10) Pub. No.: US 2006/0240569 A1
(43) Pub. Date: Oct. 26, 2006

(54) Semi-quantitative immunochromatographic device

(75) Inventors: Paul E. Goldenbaum, Hampstead, MD (US); Stephen J. Lovell, Lutherville, MD (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(73) Assignee: Becton, Dickinson and Company, Franklin Lakes, NJ (US)

(21) Appl. No.: 11/341,723

(22) Filed: Jan. 27, 2006

Related U.S. Application Data
(60) Provisional application No. 60/673,218, filed on Apr. 20, 2005.

(51) Int. Cl. G01N 33/558

(52) U.S. Cl. 436/514

(57) ABSTRACT

The invention provides a device comprising one or more support materials capable of providing lateral flow. The one or more support materials contain an area for a receiving a biological sample containing a target analyte, an area having a movably contained detector ligand capable of forming a complex with the target analyte, a first capture area having a predetermined amount of an immobile capture reagent, where the immobile capture reagent is capable of specifically binding to the complex, a second capture area having the immobile capture reagent, and a lysing agent. Typically, the area for receiving the biological sample, the area having the movably contained detector ligand, the first capture area, and the second capture area are arranged in the one or more support materials such that a sample is capable of sequential lateral flow through these areas.

### RELATED APPLICATIONS

This application claims priority to U.S. Provisional Patent Application Ser. No. 60/673,218 filed Apr. 20, 2005. The disclosure of which is hereby incorporated by reference.

### FIELD OF INVENTION

The present invention is directed to devices and methods for the semi-quantitative analysis of a sample for a target analyte.

### BACKGROUND OF THE INVENTION

In the following discussion certain articles and methods will be described for background and introductory purposes. Nothing contained herein is to be construed as an “admission” of prior art. Applicant expressly reserves the right to demonstrate, where appropriate, that the articles and methods referenced herein do not constitute prior art under the applicable statutory provisions.

Various immunological reactions have been found useful as analytical techniques. Immunochemical assays generally fall into a number of different categories. Two of the most frequently encountered types are the competitive assay, and the sandwich assay. In a competitive assay, a limited quantity of binding material is contacted with a solution containing an analyte and a known concentration of a labeled analyte. The labeled and unlabeled analyte compete for binding sites on a binding material. The amount of labeled analyte bound to the binding material can be correlated with the concentration of the analyte present in the test solution. Quantification is typically accomplished by reference to a calibration curve, visual comparison of the extent of color change caused by the reaction, or evaluation with a testing instrument.

The sandwich assay involves contacting a binding material with a solution containing the analyte, thereby causing the analyte to bind to a binding material. This complex is then contacted with a labeled binding material, generally an antibody, which reacts with the bound analyte. The amount of bound labeled binding material is thus directly proportional to the amount of bound analyte. Quantification is typically accomplished by comparison of the color change caused by the reaction with a standard or reference, reference to a calibration curve, or inspection by a testing instrument.

Conventional techniques, and devices that rely on such techniques, however, have demonstrated various deficiencies. For example, while relatively simple test strip devices have been developed, they frequently lack the capability of at least immediately quantifying the results of the assay. In this regard, many such devices simply indicate a positive or negative result for the presence of the analyte above or below a chosen threshold value. Moreover, such devices typically require a washing step, or they require the mixing of one or more of the reagents and the sample. The requirement to perform such washing or mixing steps introduces an undesirable complexity to the assay, which does not render it desirable for use by laypersons or even lower level medically trained personnel.

Improvements in, and new applications for, such tests are desired.

### SUMMARY OF THE INVENTION

According to a first aspect, the invention provides a device comprising one or more support materials capable of providing lateral flow. The one or more support materials contain an area for a receiving a biological sample containing a target analyte, an area having a movably contained detector ligand capable of forming a complex with the target analyte, a first capture area having a predetermined amount of an immobile capture reagent, where the immobile capture reagent is capable of specifically binding to the complex, a second capture area having the immobile capture reagent, and a lysing agent. Typically, the area for receiving the biological sample, the area having the movably contained detector ligand, the first capture area, and the second capture area are arranged in the one or more support materials such that a sample is capable of sequential lateral flow through these areas.

According to another aspect, the target analyte is CD4 antigen present in CD4 lymphocytes. Thus, the one or more materials contain an area having a movably contained detector ligand capable of forming a complex with the CD4 antigen, a first capture area having a predetermined amount of an immobile capture reagent capable of specifically binding to the complex, and a second capture area comprising the immobile capture reagent. Typically, the area for receiving the biological sample, the area having the movably contained detector ligand, the first capture area, and the second capture area are arranged in the one or more support materials such that a sample is capable of sequential lateral flow through these areas.

The invention further provides methods of using such devices.

### BRIEF DESCRIPTION OF THE DRAWINGS

The foregoing and other features, aspects and advantages of the present invention will become apparent from the following description, appended claims and the exemplary embodiments shown in the drawings, which are briefly described below. It should be noted that, unless otherwise specified, like elements have the same reference numbers.

FIG. 1 is perspective view of a device constructed according to the principles of the present invention.

FIG. 2 is a perspective view of a device constructed according to an alternative embodiment of the present invention.

FIGS. 3A-3D are perspective views of devices constructed according to further embodiments of the present invention.

FIG. 4 is still another embodiment of a device constructed according to the principles of the present invention.

### DETAILED DESCRIPTION OF THE INVENTION

The principles of the present invention will now be further described by the following discussion of certain illustrative embodiments thereof and by reference to the foregoing drawing figures. Unless otherwise indicated herein, the same drawing reference numbers or characters have been utilized in multiple drawing figures to indicate the illustration of the same or similar elements.

The term “analyte”, as used herein, refers to a compound or composition to be detected or measured in the test sample. The analyte will have at least one epitope that an antibody or an immunological reactive fragment thereof can recognize. Analyte can include any antigenic substances, haptens, antibodies and combinations thereof. The target analyte of interest in an assay can be, for example, a protein, a peptide, an amino acid, a nucleic acid, a hormone, a steroid, a vitamin, a pathogenic microorganism for which polyclonal and/or monoclonal antibodies can be produced, a natural or synthetic chemical substance, a contaminant, a drug including those administered for therapeutic purposes as well as those administered for illicit purposes, and metabolites of or antibodies to any of the above substances. One specific example comprehended by this term is CD4 antigen present in CD4 lymphocytes.

The term “biological sample” as used herein refers to any sample that could contain an analyte for detection. Preferably, the biological sample is in liquid form or can be changed into a liquid form. The biological sample can comprise whole blood, urine, saliva, or other bodily fluids and secretions. The term biological sample is not specific as to its source. Thus the sample may be obtained from any source, such as humans or other mammals.

As used herein, the term “sample receiving area” means the portion of an assay device that is first contacted with the biological sample, i.e., it receives the sample to be tested for the analyte in question.

The term “lateral flow” refers to flow in which the components of the sample are carried through the material in the lateral direction. For example, when the assay device is in the form of a sheet or strip, the lateral direction is parallel to the plane defined by the major surface(s) of the device.

As used herein, the term “material” refers to any substance capable of providing lateral flow. This would include materials such as cellulose esters, nitrocellulose, nitrocellulose blends with polyester or cellulose, untreated paper, porous paper, porous polyethylene, porous polypropylene, rayon, glass fiber, acrylonitrile copolymer or nylon. One skilled in the art will be aware of other porous materials that allow lateral flow.

The term “mobile,” as referred to herein, means diffusively or non-diffusively attached, or impregnated. Substances or reagents, which are mobile, are capable of dispersing with the sample and being carried by the lateral flow.

The term “immobile,” as used herein refers to substances or reagents, which are attached to a support material such that lateral flow of the liquid sample does not affect the placement of the immobile particle in the material. Such attachment can be, for example, through covalent or ionic means. Those skilled in the art will be aware of means of attachment to immobilize various substances or reagents.

The term “detector ligand” as used herein refers to any particle, protein or molecule which recognizes or binds to the analyte in question, and has attached conjugated or bound to it, either chemically, covalently, noncovalently, ionicly or nonionicly any substance capable of producing a detectable signal. Such substances capable of producing a detectable signal would include chromogens, catalysts, fluorescent compounds, colloidal metallic and/or nonmetallic particles, dye particles, enzymes or substrates, organic polymers, latex particles, liposomes with signal producing substances of the type described in U.S. Pat. No. 4,703,017, the entire content of which is incorporated herein by reference. The particle or molecule recognizing the analyte can be either natural or non-natural. According to exemplary embodiments, the detector ligand comprises a ligand conjugated to a colloidal gold particle, or to a liposome containing a visible dye.

The term “detectable signal”, as used herein refers to any indicator that is visible with the naked eye and/or an optical reading instrument on which can be translated into such a visual indicator. “Visible to the naked eye” is intended to encompass the use of corrective lenses and magnification means. The detectable signal may be immediately and continuously visible, or can become visible only upon reaction with another reagent. For example, colloidal metals such as gold are typically immediately and continuously visible. Alternatively, the substance capable of producing a signal can comprise a first reactant such as an enzyme, and a second reactant can be provided in a separate area, such that upon migration of the enzyme to the separate area a chemical reaction occurs and a change is color results in the separate area.

The term “detector ligand area,” as used herein refers to an area of the device wherein mobile detector ligand is disposed.

The term “control reagent” as used herein refers to any particle or molecule, which is capable of binding the ligand detector, but does not recognize or bind the target analyte. For example, the detector ligand can be a ligand conjugated to a substance capable of producing a signal. The control reagent would be a particle or molecule, which recognizes or binds the ligand portion of the detector ligand when the analyte is not bound thereto. Preferably, the control reagent would be a monoclonal or polyclonal antibody, which recognizes the ligand. The control reagent is immobile. Thus, once it binds the unbound labeling reagent it immobilizes the labeling reagent and prevents it from continuing in the lateral flow.

The term “control line,” as used herein, refers to an area of the device wherein control reagent is immobilized.

The term “capture reagent” as used herein refers to any particle or molecule, which recognizes or binds to the analyte of interest or to the detector ligand bound to the analyte. The capture reagent is immobile, thus once the capture reagent binds the analyte-detector ligand complex it prevents the complex from continuing with the lateral flow. The capture reagent can comprise any suitable ligand, such as antibodies, fabs, peptides, aptamers, etc. According to one exemplary embodiment, the capture reagent comprises an antibody to CD4 antigen present in CD4 lymphocytes.

The term “capture area,” as used herein, refers to an area of the device wherein capture reagent is immobilized in the material.

The term “specifically bind,” as used herein refers to a binding mechanism which occurs via a receptor, capture agent, or the like, and which selectively couples only with a specific agent or substance.

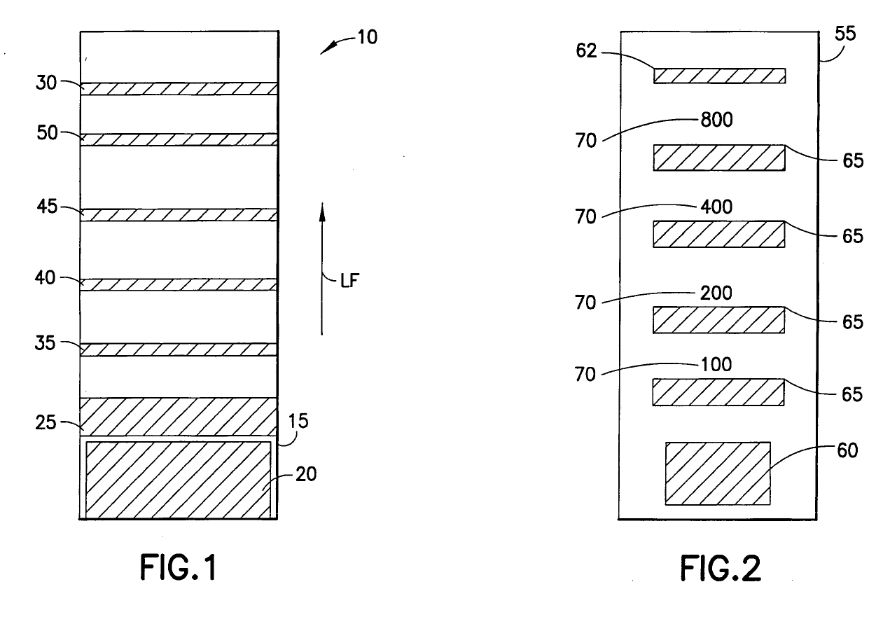

A first embodiment constructed according to the principles of the present invention will now be described by reference to FIGS. 1 and 2. The device 10 generally comprises a support material 15, which is capable of producing lateral flow, that is flow in the direction of arrow LF.

As illustrated in FIG. 1, the support material 15 can be shaped in the form of a strip, and will be referred to as such in the description that follows. Alternatively, the support material 15 can be provided in a wide variety of shapes or forms so long as the particular form permits the various functions described herein.

This support 15 can also be formed from a number of different suitable materials, provided the materials allow the aforementioned lateral flow functionality. For example, the material can comprise: glass fiber, cellulose ester, nylon, cross-linked dextran, etc. According to one embodiment, the material comprises nitrocellulose. As noted in the example below, the support can comprise one or more distinct support materials, in addition to being a single strip.

In the illustrative device 10, a biological sample receiving area 20 is provided on the strip 15. While in the illustrated embodiment only one sample receiving is area 20 is shown located at the end of the strip 15, it should be understood that it is comprehended by the present invention that the plurality of sample receiving areas can be provided. Moreover, the sample receiving area(s) can have locations which differ from that of the illustrated embodiment. The device 10 further includes an area 25 containing detector ligand. The detector ligand is contained within the area 25 in a manner such that it is mobile. In other words, the detector ligand is capable of being carried out of the area 25 by the aforementioned lateral flow. According to an illustrative, non-limiting example, 3-7 μL, preferably 5 μL, of detector ligand is applied per cm width in area 25 and dried. Detector ligand preferably comprises 10-200 μg, preferably 50 μg, of antibody conjugated per 1 mL suspension of 10-100 nm, preferably 40 nm colloidal gold particles. In the illustrated embodiment, the area 25 containing the detector ligand and the biological sample receiving area 20 are illustrated as separate and distinct areas on the strip 15. However, it is within the scope of the present invention that these two areas could be combined so as to define a single indistinct area of the strip 15.

An optional control line 30 can also be provided on the strip 15. When present, the control line 30 defines an area which contains a control reagent. The control reagent contained within the control line 30 is immobile. According to an illustrative, non-limiting example, 0.1-10 μg, preferably 1 μg, of the control reagent is disposed per cm width in the control line 30.

A first capture area 35 is provided in the illustrated embodiment, and is in lateral flow communication with the previously described areas of the strip 15. The first capture area 35 contains a capture reagent therein. The capture reagent is immobly contained within the first capture area 35. In an embodiment useful for CD4 determinations, 2 ng per cm width of capture agent is disposed in the first capture area 35 (assumes 60,000 CD4 molecules per cell, 100/L sample of blood, capture area saturated at 100 CD4 cells per μL, 5 mm width of strip, molar mass of antibody 150,000 g/mol).

According to another optional feature of the present invention, a lysing agent may be added to the strip material 15 at various locations. For example, the lysing agent may be contained within the sample receiving area 20 or any area of the strip 15 located between the sample receiving area 20 and the first capture area 35. Suitable lysing agents include, for example, zwittergent 3-10 and 3-14, saponin, Triton X100, Tween 20, SDS (sodium dodecyl sulfate) and CTAB.

According to the illustrated embodiment, second, third and fourth capture areas 40, 45 and 50 respectively, are additionally provided on the strip 15. However, it should be understood that it is within scope of the present invention for the strip 15 to contain at least one capture area. According to a further embodiment, the strip 15 may include at least two capture areas. According to a further embodiment, the strip 15 may include at least four capture areas.

According to the illustrated embodiment, the capture areas are distinct and separated from one another in the direction of lateral flow LF. However, it is comprehended by the present invention that one or more capture areas can be combined and/or otherwise merged. Additionally, location, shape, size and configuration of the capture areas may also deviate from that of the illustrated embodiment.

The embodiment depicted in FIG. 2 essentially corresponds to the embodiment of FIG. 1, but further includes an optional housing 55. The housing 55 can be formed of any suitable material. For example, the housing may be formed of a plastic material, such as Mylar. The housing 55 at least partially encloses or surrounds the strip 15. As illustrated in FIG. 2, the housing may be provided with a sample receiving area window 60, a control line window 62, and capture area windows 65. According to the illustrated embodiment, the area containing the detector ligand is obscured from view of the user by the housing 55. It is of course comprehended that the area containing the detector ligand may be visible through the housing to the user as well. Moreover, the housing 55 can be formed from a clear or translucent plastic material.

The housing 55 can optionally be provided with indicia 70 which identify various degradations of the concentration of target analyte determined to be present in the biological sample by the device 10. As evident from the indicia 70 illustrated in FIG. 2, it is possible to ascertain, within a particular range of values, the concentration of an analyte present in a biological sample, as described in further detail herein.

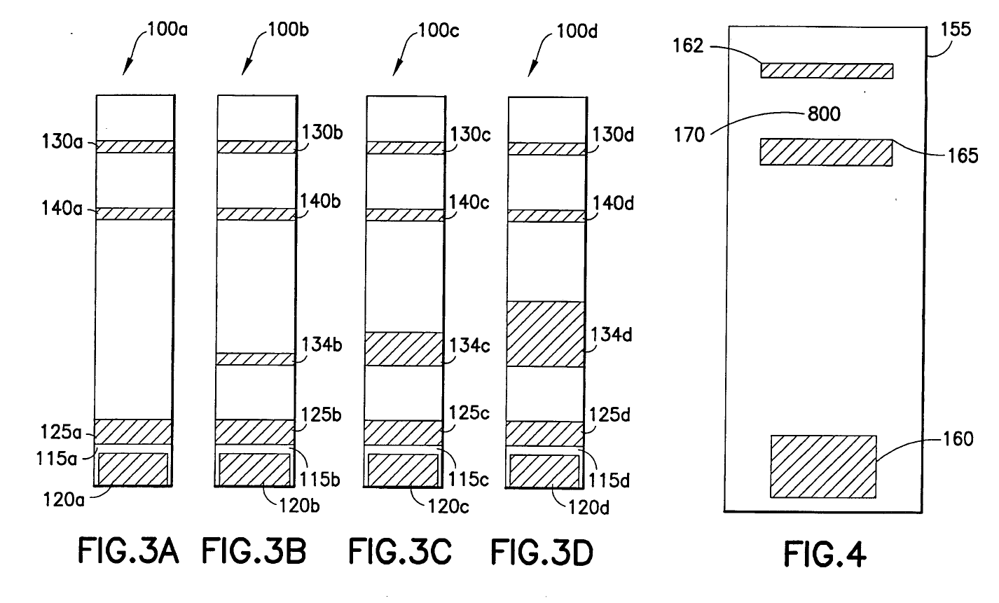

The embodiments illustrated in FIGS. 3A-3D contain many of the features described in connection with the embodiments illustrated in FIGS. 1 and 2. Generally speaking, the embodiments illustrated in FIG. 3 differ from the previously-described embodiments in that each strip contains a single capture area for indicating a threshold concentration level of target analyte. A positive indication is given in the capture area only when a specified, predetermined concentration of analyte is present in the biological sample.

Thus, the previous description, as well as the description which follows, applies equally to the strips illustrated in FIGS. 3A-3D.

According the illustrated embodiment of FIG. 3A, a device 100a is provided by support material 115a generally formed as a strip. The strip 115a includes a sample receiving area 120a, an area containing detector ligand 125a, and an optional control line 130a. The detector ligand contained in the area 125a is mobile, and thus capable of being transported out of area 125a by lateral flow. The control line 130a defines an area containing at least one control reagent. The control reagent is immobly contained within the control line 130a. A first capture area 134a is illustrated as being separate from, yet in lateral flow communication with the aforementioned areas of the strip 115a. It is of course, as explained in connection with the previous embodiments, that the location number and arrangement of the various areas of the strip 115a may vary from that of the illustrated embodiment. Thus, reference is made to the previous description made in connection with FIGS. 1 and 2. The capture area 140a contains an immobile capture reagent provided in a predetermined specified amount.

As illustrated in FIGS. 3B-3D, the strip 115b-d may be provided with both a first and second capture areas 134b-d and 140b-d, respectively. The amount of capture reagent provided in the capture area 134b-d is used to capture a predetermined specific amount of target analyte contained in the biological sample. The first capture area 134b-d is not utilized to provide any indication, per se, with regard to the amount of target analyte contained in the sample. According to the illustrated embodiment, a second capture area 140b-d is provided for this purpose. The second capture area 140b-d also contains a specific predetermined amount of capture reagent therein. The capture reagent is also immobly contained within the second capture area 140b-d. However, it is within the scope of the present invention that the number, location, and arrangement of the capture areas can vary.

FIG. 4 is illustrative of an additional embodiment of the present inventions wherein the device 100 has the same features described above in connection with the embodiments of FIGS. 3A-3D, but additionally contains an optional housing member 155. The housing 155 can be constructed of any suitable material. For example, the housing 155 can be constructed of a plastic material, such as Mylar. The optional housing 155 can be provided with a sample receiving area window 160, a control line window 162, a capture area window 165 and an indicia 170. According to the illustrated embodiment, the first capture area is obscured from view by the housing 155. Where the first capture area 134 provides no useful indication with regard to the amount of analyte contained in the biological sample, it is not necessary that this area be visible to the user. However, it is comprehended that the housing 155 could be modified such that at least this additional area of the strip 115 can be made visible to the user. Alternatively, the housing 155 can be constructed of a clear or translucent plastic material.

The above-described devices can be constructed by utilizing techniques familiar to those skilled in the art. The above-described reagents are optionally prepared in a liquid form for deposit into and/or upon the support material. Once placed in contact with the support material, the liquid reagent dries and adheres to the support material. Conventional fillers, binders, surfactants and the like may be incorporated into the liquid reagent if so desired. In addition, binder materials may also be incorporated into the liquid reagent compositions to facilitate adhesion to the support material.

It is desirable to accurately control the volume of reagents introduced into the support material in any particular area. Thus, the use of pipettes, micropipettes and the like to control the volume of reagent introduced is comprehended by the present invention. In addition, it may be desirable to isolate or otherwise confine the area into which a particular reagent is introduced. This area can be controlled according to techniques known to those skilled in the art. For example, it is within the scope of the present invention that one or more of the previously described areas of the strip of the present invention can be separated by areas that are non-absorbent and/or hydrophobic. This separation may be accomplished by treating the support material in these intervening areas, or alternatively, introducing sections or segments of a different material that imparts these desired properties.

As evident from the above-description of the embodiment depicted in FIGS. 1-4, it is desirable to introduce certain reagents into the support material in a manner that renders them immobile therein. The immobilization can be accomplished by any convenient technique such as evaporative deposition, adsorption, covalent bonding or immunological immobilization. Such techniques are described in greater detail, for example, in U.S. Pat. Nos. 4,517,288 and 4,186,146, the entire content of which is incorporated herein by reference in its entirety. It is also desirable to be able to introduce certain reagents into the support material in a manner such that they remain mobile therein. Such an application may be accomplished by simply pipetting small volumes of liquid reagent solutions to the desired areas. Such techniques are familiar to those skilled in the art.

According to a further aspect of the present invention, a method or technique for assaying a target analyte will now be described. For purposes of illustration, reference will be made to the illustrative embodiments of FIGS. 1-4. However, it should be understood that the method and techniques of the present invention are not necessarily limited by the physical constructions and arrangements of the embodiments illustrated in FIGS. 1-4.

An illustrative embodiment of an assay conducted according to the principles of the present invention will be described in further detail as follows, and will be described for purposes of illustration as a sandwich-type assay. However, it is comprehended by the present invention that one of ordinary skill in the art, armed with the teachings contained herein, could accomplish the same objectives through performance of competition-type and/or inhibition-type assays. Thus, the method of the present invention should not necessarily be construed as limited to a sandwich-type assay.

An assay performed according to the present invention begins with collection of an appropriate biological sample (for convenience only, references to FIGS. 1 and 3B are provided). An appropriate sample volume can vary widely. For example, a sample on the order 1-100 μl can be utilized. According to one embodiment of the present invention, the biological sample comprises whole blood. The biological sample is added to a sample receiving area (20, 120b) of a test strip (15, 115b). The test strip material promotes lateral flow (LF) of the biological sample within the material. The biological sample flows into an area containing a detector ligand (25, 125b). The detector ligand is designed to specifically bind to the particular target analyte under investigation. Thus, as target analyte travels by lateral flow through the detector ligand containing area, the target analyte becomes specifically bound thereto and travels, as a complex, out of the aforementioned area.

Optionally, the biological sample containing the aforementioned complex is carried by the lateral flow into a control line area containing a control reagent (30, 130b). In this region, the control line area serves as an internal procedural control and the detection of signal in this area verifies that capillary flow has taken place and that the functional integrity of the device was maintained.

The biological sample and complex contained therein, then laterally flows to a first capture area (35, 134b). The first capture area contains an immobile capture reagent configured to specifically bind to the complex containing the target analyte. Thus, as the complex travels through the first capture area, it becomes immobly bound to the capture reagent. To the extent that the biological sample contains an amount of target analyte such that the capacity of the capture reagent contained in the first capture area is exceeded, any such excess target analyte, and complex formed thereby, continues to travel under the influence of the lateral flow thereby reaching one or more optionally provided additional capture areas.

As evident from the above, controlling the amount of capture reagent contained in the first capture area can be utilized to define a barrier beyond which an amount or concentration of target analyte contained in a biological sample may not pass. To the extent this minimum threshold value is exceeded, excess target analyte is free to travel under the lateral flow to one or more capture areas which again establish increasing minimum threshold levels of target analyte under analysis. In this regard, the amount of capture reagent provided in each capture area can be the same amount, relative to one another. Alternatively, the amount of capture reagent contained in each capture area can progressively increase or decrease. According to a non-limiting illustrative example, 2 ng of capture reagent can be provided in a first capture area, 2 ng of capture reagent can be provided in a second capture area, 4 ng of capture reagent can be provided in a third capture area, and 8 ng of capture reagent can be provided in a fourth capture area.

The presence of a sufficient amount of target analyte, and the complex formed from the target analyte and detector ligand, in the above-described one or more capture areas is indicated by generation of a detectable signal. This detectable signal can be generated in a number of different ways familiar to those skilled in the art. According to one example, the detector ligand can comprise a substance that is immediately and continuously visible to the naked eye. Thus, the mere physical presence of the complex formed between the target analyte and the detector ligand on the one or more capture areas is sufficient to produce the desired detectable signal. Alternatively, the detector ligand can comprise a first reactant which becomes associated with the complex formed with the target analyte, but which is not visible. A second reactant can then be provided in the one or more capture areas which, upon combination and interaction with the first reactant produces a detectable signal. The detectable signal may be visible to the naked eye, or may be read or analyzed with the assistance of a separate device such as a spectrometer, fluorimeter, microscope or the like. As a further optional aspect of an assay performed according to the principles of the present invention, a lysing agent may be introduced into the strip material, which interacts with the biological sample, thereby releasing target analyte substances. As readily apparent, the devices and methods of the present invention are useful in a number of different applications, and for a number of different purposes. In general, the devices and methods of the present invention will be useful for a semi-quantitative analysis of the amount target analyte contained in a biological sample when it is desirable to be ascertained in a simple, accurate, quick and cost effective manner. Suitable, non-limiting, applications may include blood glucose monitoring, as well as the diagnosis and treatment of viral diseases such as HIV/AIDS. It has been observed that a decline in CD4 cell counts is an effect of HIV, and that CD4 cell depletion is indicative of immune deficiency. In this regard, the Centers for Disease Control (CDC) has devised the classification system for HIV infection that emphasizes the clinical importance of CD4 cell counts. The CDC's classification system is based upon studies showing a strong association between the development of life-threatening opportunistic illnesses and CD4 cell counts. In this regard, as the CD4 cell count decreases, the risk and severity of opportunistic illnesses increased. Thus, CD4 cell counts are used to guide clinical and therapeutic management of HIV-infected persons. The CDC suggests that antiretroviral therapy should be considered for all persons with a CD4 count of less than 500 cells/μl, and that prophylactic treatment against Pneumocystis Carrinii Pneumonia (PCP) is recommended for all patients with a CD4 cell count of less than 200 cells/μl.

The three CDC categories for CD4 lymphocyte cell counts are as follows:

Category 1: Greater than or equal to 500 cells/μl;

Category 2: 200-499 cells/μl; and

Category 3: Less than 200 cells/μl.

As readily apparent from the above, the device and techniques of the present invention may be utilized to determine which CDC category a patient's CD4 cell count falls into. For example, according to the present invention, a first capture area may be provided with a specific volume of capture agent such that a detectable signal will be produced in the first capture area only if the CD4 cell count present in the biological sample is approximately 100 cells/μl or greater. A second capture area may be provided such that a detectable signal will be produced therein only upon the presence of a CD4 cell concentration of approximately 200 cells/μl or greater. A third capture area may be provided with an appropriate amount of capture reagent contained therein such that a detectable signal will be produced in the third capture area only upon the presence of approximately 400 CD4 cells/μl in the biological sample. A fourth capture area may be provided with an appropriate amount of capture reagent such that a detectable signal will be produced only upon the presence of approximately 800 CD4 cells/μl. It is within the scope of the present invention that the specified concentrations and/or numbers of capture areas provided may be altered as desired to achieve a particular diagnostic objective. CD4 cell counts are widely recognized as an important diagnostic tool in the process of determining when a patient should begin antiviral therapies, as well as an important tool in the monitoring of the efficiency and effectiveness of such antiviral therapies. Thus, frequent determination of CD4 cell counts should be made. The devices and methods of the present invention provide a simple, accurate, quick and cost effective means of frequently determining CD4 cell counts in a semi-quantitative manner.

Specific non-limiting examples comprehended by the principles of the present invention will now be described to further illustrate the concepts of the present invention.

### EXAMPLE 1

In this example, human blood can be tested for a semi-quantitative amount of CD4 lymphocytes. A drop of blood from the test subject is obtained by finger prick with lancet and applied to the area for receiving a biological sample on the lateral flow device. This is followed by adding two drops of a suitable buffer, such as phosphate buffered saline, to the receiving area in order to promote lateral flow. The device consists of a pad, for example rayon or glass fiber to receive the blood sample. A lysing agent is impregnated into the pad to lyse the blood cells. Also in this same pad is the detector antibody (monoclonal antibody specific to CD4 antigen) which is conjugated to colloidal gold. Underlying this pad is a strip of nitrocellulose, typically 5 mm wide and 30 mm in length. The pad is located at one extreme end of this strip. At the other extreme end of the strip, an absorbent pad is in contact with the strip to facilitate and maintain the capillary flow of liquid. Fluid in the blood sample will move up the strip (“upstream”) as a result of capillary action. Deposited immovably on the nitrocellulose strip in this example are four zones of capture antibody which are capable of forming a sandwich complex with the CD4-detector complex. The capture zone closest to the sample deposition area contains enough capture antibody to become saturated with CD4 antigen from the equivalent of 100 CD4 lymphocytes per μL of sample. The second, third and fourth capture zones would similarly contain capture antibody to become saturated with the equivalents of 200, 400 and 800 CD4 lymphocytes per μL of sample, respectively. The strip is allowed to develop for 15 minutes at which time the number of visible bands on the strip are counted. The readibility of the test strip can be enhanced by applying a wash solution after the blood sample to clear out any background color caused by hemoglobin. The number of bands indicates the number of CD4 cells in the sample of blood. If only the first band is observed the level of CD4 cells is 50-100 cells/μL. If only the first and second bands are observed, the level is approximately 200 cells/μL. If the first three bands are observed, the level is approximately 400 cells/μL and if all four bands are observed, the level is greater than 800 cells/μL.

While this invention is satisfied by embodiments in many different forms, as described in detail in connection with preferred embodiments of the invention, it is understood that the present disclosure is to be considered as exemplary of the principles of the invention and is not intended to limit the invention to the specific embodiments illustrated and described herein. Numerous variations may be made by persons skilled in the art without departure from the spirit of the invention.

We claim:

1. A device comprising:
one or more support materials capable of providing lateral flow, the one or more support materials comprising:
an area for a receiving a biological sample containing a target analyte;
an area comprising a movably contained detector ligand, wherein the detector ligand is capable of forming a complex with the target analyte, and wherein the area comprising the movably contained detector ligand and the area for receiving the biological sample may be separate areas, the same area, or partially the same area;
a first capture area comprising a predetermined amount of an immobile capture reagent, the immobile capture reagent capable of specifically binding to the complex;
a second capture area comprising the immobile capture reagent; and
a lysing agent.

2. The device of claim 1, wherein the area for receiving the biological sample, the area comprising the movably contained detector ligand, the first capture area, and the second capture area are arranged in the one or more support materials such that a sample is capable of lateral flow sequentially through the area for receiving the biological sample, the area comprising the movably contained detector ligand, the first capture area, and the second capture area.

3. The device of claim 1, wherein the one or more support materials comprise a cellulose ester, DEAE, glass, nylon, particulate silica, polystyrene, polyethylene, polyamide, polyacrylamide, polyvinyl, polypropylene, cellulose agarose, or dextran.

4. The device of claim 1, wherein the one or more support materials comprises a single strip comprising the area for receiving the biological sample, the area comprising the movably contained detector ligand, the first capture area, the second capture area, and the lysing agent.

5. The device of claim 1, wherein the target analyte is CD4 antigen present in CD4 lymphocytes.

6. The device of claim 1, wherein the immobile capture reagent comprises an antibody capable of specifically binding to the CD4 antigen present in CD4 lymphocytes.

7. The device of claim 1, wherein the detector ligand comprises colloidal gold conjugated to an antibody.

8. The device of claim 1, wherein the predetermined amount present in the first capture area is sufficient to bind the complex at a specified level.

9. The device of claim 8, wherein the second capture area comprises a predetermined amount of the immobile capture reagent sufficient to bind the complex at a second specified level.

10. The device of claim 9, wherein the second capture area is capable of indicating the presence of a least a second amount of the target analyte present in the sample.

11. The device of claim 10, wherein the one or more support materials further comprise a third capture area comprising a predetermined amount of the immobile capture reagent.

12. The device of claim 11, wherein the one or more support materials further comprise a fourth capture area comprising a predetermined amount of the immobile capture reagent.

13. The device of claim 12, wherein the third capture area is capable of indicating the presence of a least a third amount of target analyte present in the sample, and the fourth capture area is capable of indicating the presence of at least a fourth amount of target analyte present in the sample, wherein the third amount is greater than the second amount, and the fourth amount is greater than the third amount.

14. The device of claim 1, further comprising:
a control line containing an immobile control reagent.

15. The device of claim 13, wherein the target analyte is CD4 antigen present in CD4 lymphocytes, the movably contained detector ligand is capable of forming the complex with the CD4 antigen, the immobile capture reagent specifically binds the complex, and the first area is capable of indicating the presence of at least approximately 100 CD4 cells/μl in the sample.

16. The device of claim 15, wherein the second capture area is capable of indicating the presence of at least approximately 200 CD4 cells/μl in the sample.

17. The device of claim 16, wherein the third capture area is capable of indicating the presence of at least approximately 400 CD4 cells/μl in the sample.

18. The device of claim 17, wherein the fourth capture area is capable of indicating the presence of at least approximately 800 CD4 cells/μl in the sample.

19. The device of claim 1, further comprising a housing at least partially covering the one or more support materials.

20. The device of claim 1, wherein the lysing agent is located in the sample receiving area.

21. The device of claim 1, wherein the lysing agent is located laterally between the sample receiving area and the first capture area, such that a sample flowing laterally from the sample receiving area will be exposed to the lysing agent prior to the sample reaching the first capture area.

22. A device comprising:
one or more support materials capable of providing lateral flow, the one or more support materials comprising:
an area for a receiving a biological sample containing CD4 antigen present in CD4 lymphocytes;
an area comprising a movably contained detector ligand, wherein the detector ligand is capable of forming a complex with the CD4 antigen, and wherein the area comprising the movably contained detector ligand and the area for receiving the biological sample may be separate areas, the same area, or partially the same area;
a first capture area comprising a predetermined amount of an immobile capture reagent, the immobile capture reagent capable of specifically binding to the complex; and
a second capture area comprising the immobile capture reagent.

23. The device of claim 22, wherein the area for receiving the biological sample, the area comprising the movably contained detector ligand, the first capture area, and the second capture area are arranged in the one or more support materials such that a sample is capable of lateral flow sequentially through the area for receiving the biological sample, the area comprising the movably contained detector ligand, the first capture area, and the second capture area.

24. The device of claim 22, wherein the one or more support materials comprise a cellulose ester, DEAE, glass, nylon, particulate silica, polystyrene, polyethylene, polyamide, polyacrylamide, polyvinyl, polypropylene, cellulose agarose, or dextran.

25. The device of claim 22, wherein the one or more support materials comprises a single strip comprising the area for receiving the biological sample, the area comprising the movably contained detector ligand, the first capture area, and the second capture area.

26. The device of claim 22, wherein the immobile capture reagent comprises an antibody capable of specifically binding to the CD4 antigen present in CD4 lymphocytes.

27. The device of claim 22, wherein the detector ligand comprises colloidal gold conjugated to an antibody which recognizes CD4 antigen and is capable of forming a sandwich complex with the immobile capture reagent and the CD4 antigen.

28. The device of claim 22, wherein the second capture area comprises a predetermined amount of the immobile capture reagent, and is capable of indicating the presence of at least a second amount of CD4 antigen present in the sample.

29. The device of claim 28, wherein the one or more support materials further comprise a third capture area comprising a predetermined amount of the immobile capture reagent.

30. The device of claim 29, wherein the one or more support materials further comprise a fourth capture area comprising a predetermined amount of the immobile capture reagent.

31. The device of claim 30, wherein the third capture area is capable of indicating the presence of a least a third amount of CD4 antigen present in the sample, and the fourth capture area is capable of indicating the presence of at least a fourth amount of CD4 antigen present in the sample, wherein the third amount is greater than the second amount, and the fourth amount is greater than the third amount.

32. The device of claim 22, further comprising:
a control line containing an immobile control reagent.

33. The device of claim 22, wherein the first capture area is capable of indicating the presence of at least approximately 100 CD4 cells/μl in the sample.

34. The device of claim 28, wherein the second capture area is capable of indicating the presence of at least approximately 200 CD4 cells/μl in the sample.

35. The device of claim 31, wherein the third capture area is capable of indicating the presence of at least approximately 400 CD4 cells/μl in the sample.

36. The device of claim 35, wherein the fourth capture area is capable of indicating the presence of at least approximately 800 CD4 cells/μl in the sample.

37. The device of claim 22, further comprising a housing at least partially covering the one or more support materials.

38. A method for determining the amount of a target analyte in a biological sample, comprising the steps of:
providing a device comprising:
one or more support materials capable of providing lateral flow, the one or more support materials comprising:
an area for a receiving the biological sample;
an area comprising a movably contained detector ligand, wherein the detector ligand is capable of forming a complex with the target analyte, and wherein the area comprising the movably contained detector ligand and the area for receiving the biological sample may be separate areas, the same area, or partially the same area;
a first capture area comprising a predetermined amount of an immobile capture reagent, the immobile capture reagent capable of specifically binding to the complex;
a second capture area comprising the immobile capture reagent; and
a lysing agent;
disposing the sample onto the area for receiving the biological sample; and
using visual indicators provided by the detector ligand to determine information regarding the amount of the target analyte in the sample.

39. The method of claim 38, wherein the area for receiving the biological sample, the area comprising the movably contained detector ligand, the first capture area, and the second capture area are arranged in the one or more support materials such that the biological sample sequentially and laterally flows through the area for receiving the biological sample, the area comprising the movably contained detector ligand, the first capture area, and the second capture area.

40. The method of claim 38, wherein the target analyte is CD4 antigen present in CD4 lymphocytes, and the capture reagent comprises an antibody capable of specifically binding to the CD4 antigen presenting CD4 lymphocytes.

41. The method of claim 38, wherein the predetermined amount present in the first capture area is sufficient to bind the complex at a specified level.

42. The method of claim 38, wherein the second capture area comprises a predetermined amount of the immobile capture reagent sufficient to bind the complex at a second specified level, and wherein the detector ligand at the second capture area indicates the presence of at least a second amount of the target analyte in the sample.

43. The method of claim 42, wherein the one or more support materials further comprise a third capture area comprising a predetermined amount of the immobile capture reagent, wherein the one or more support materials further comprise a fourth capture area comprising a predetermined amount of the immobile capture reagent, and wherein the third capture area is capable of indicating the presence of at least a third amount of target analyte present in the sample, and the fourth capture area is capable of indicating the presence of at least a fourth amount of target analyte present in the sample, wherein the third amount is greater than the second amount, and the fourth amount is greater than the third amount.

44. The method of claim 38, wherein the target analyte is CD4 antigen present in CD4 lymphocytes, the movably contained detector ligand is capable of forming the complex with the CD4 antigen, the immobile capture reagent specifically binds the complex, and the first area is capable of indicating the presence of at least approximately 100 CD4 cells/μl in the sample.

45. The method of claim 44, wherein the second capture area is capable of indicating the presence of at least approximately 200 CD4 cells/μl in the sample.

46. The method of claim 45, wherein the third capture area is capable of indicating the presence of at least approximately 400 CD4 cells/μl in the sample.

47. The method of claim 46, wherein the fourth capture area is capable of indicating the presence of at least approximately 800 CD4 cells/μl in the sample.

48. A method for determining the amount of CD4 lymphocytes in a biological sample, comprising the steps of:
providing a device comprising:
one or more support materials capable of providing lateral flow, the one or more support materials comprising:
an area for a receiving the biological sample;
an area comprising a movably contained detector ligand, wherein the detector ligand is capable of forming a complex with a CD4 antigen on the CD4 lymphocytes, and wherein the area comprising the movably contained detector ligand and the area for receiving the biological sample may be separate areas, the same area, or partially the same area;
a first capture area comprising a predetermined amount of an immobile capture reagent, the immobile capture reagent capable of specifically binding to the complex; and
a second capture area comprising the immobile capture reagent;
disposing the sample onto the area for receiving the biological sample; and
using visual indicators provided by the detector ligand to determine information regarding the amount of the CD4 lymphocytes in the sample.

49. The method of claim 38, wherein the area for receiving the biological sample, the area comprising the movably contained detector ligand, the first capture area, and the second capture area are arranged in the one or more support materials such that the biological sample sequentially and laterally flows through the area for receiving the biological sample, the area comprising the movably contained detector ligand, the first capture area, and the second capture area.

50. The method of claim 38, wherein the target analyte is CD4 antigen present in CD4 lymphocytes, and the capture reagent comprises an antibody capable of specifically binding to the CD4 antigen presenting CD4 lymphocytes.

51. The method of claim 48, wherein the predetermined amount present in the first capture area is sufficient to bind the complex at a specified level.

52. The method of claim 48, wherein the second capture area comprises a predetermined amount of the immobile capture reagent sufficient to bind the complex at a second specified level, and wherein the detector ligand at the second capture area indicates the presence of at least a second amount of the CD4 lymphocytes in the sample.

53. The method of claim 52, wherein the one or more support materials further comprise a third capture area comprising a predetermined amount of the immobile capture reagent, wherein the one or more support materials further comprise a fourth capture area comprising a predetermined amount of the immobile capture reagent, and wherein the third capture area is capable of indicating the presence of at least a third amount of CD4 lymphocytes present in the sample, and the fourth capture area is capable of indicating the presence of at least a fourth amount of CD4 lymphocytes present in the sample, wherein the third amount is greater than the second amount, and the fourth amount is greater than the third amount.

54. The method of claim 48, wherein the first area is capable of indicating the presence of at least approximately 100 CD4 cells/μl in the sample.

55. The method of claim 52, wherein the second capture area is capable of indicating the presence of at least approximately 200 CD4 cells/μl in the sample.

56. The method of claim 55, wherein the third capture area is capable of indicating the presence of at least approximately 400 CD4 cells/μl in the sample.

57. The method of claim 56, wherein the fourth capture area is capable of indicating the presence of at least approximately 800 CD4 cells/μl in the sample.
